(19) United States
(12) Patent Application Publication
(10) Pub. No.: US 2026/0283864 A1
(43) Pub. Date: Sep. 24, 2026

(54) PANT PRODUCTS AND SYSTEM AND METHOD FOR FORMING SAME

(71) Applicant: CURT G. JOA, INC., Sheboygan Falls, WI (US)

(72) Inventors: Jeffrey W. Fritz, Plymouth, WI (US); Lloyd F. Kreif, Sheboygan Falls, WI (US); David E. Schuette, Kiel, WI (US); Scott R. Kroeplien, Sheboygan Falls, WI (US); Christopher A. Schwartz, Sheboygan Falls, WI (US); Samuel E. Darcy, Sheboygan Falls, WI (US)

(21) Appl. No.: 19/473,609

(22) PCT Filed: Apr. 10, 2024
(86) PCT No.: PCT/US2024/023844

§ 371 Date: Oct. 8, 2025

Related U.S. Application Data
(60) Provisional application No. 63/495,603, filed on Apr. 12, 2023.

(51) Int. Cl. A61F 13/496; A61F 13/15

(52) U.S. Cl. CPC A61F 13/496; A61F 13/15593; A61F 13/15739; A61F 13/15747

(57) ABSTRACT

A disposable pant product has a first waistband bonded to a second waistband at first and second side seams. Each waistband has elastic threads between first and second web layers and bonds bonding the first and second web layers together. An absorbent core is provided between the waistbands. Each side seam has a plurality of discrete bonds which bond the first and second waistbands together. Some of the discrete bonds also anchor the elastic threads in position relative to the web layers. A system for manufacturing the pant product and method of forming same are provided.

### CROSS-REFERENCE TO RELATED APPLICATIONS

This application claims priority U.S. Provisional Application No. 63/495,603 filed Apr. 12, 2023, which is herein incorporated by reference in its entirety.

### FIELD OF THE DISCLOSURE

This disclosure generally relates to a disposable pant product having elastic strands anchored via adhesive-free welds in side seams thereof, and the system and method for forming or manufacturing same.

### BACKGROUND OF THE DISCLOSURE

Generally, disposable undergarments such as pant-type diapers (generally, “pant products”) are made up of two web layers with embedded elastic threads that form front and back waistbands and an absorbent core that is inserted onto or within the waistbands to be positioned proximate to the body of a user. Such pant products are generally manufactured in a processing line of stations that perform various functions. For example, the cores may be layered and sectioned, the waistbands may be formed from continuous webs of material onto which the cores are positioned, the waistbands are bonded to create side seams, and the continuous web of material is cut into individual products.

The elastic threads are positioned in the waistbands and may be secured thereto with adhesive or anchoring bonds formed via mechanical means (e.g., thermal, pressure, or ultrasonic). During the typical manufacturing process of an elastic waistband, the elastic threads are positioned in a tensioned state between a first web layer and a second web layer. First and second web layers are typically layers of nonwoven materials or webs. Elastic threads are positioned in groups forming waist elastic. Various anchoring means for securing the elastic threads within the waistbands and leg cuff regions are known. In some embodiments, anchoring bonds are formed when bonds are formed in the web layers on opposites sides of the elastic threads, and when the tensioned elastic threads are relaxed, the bonds frictionally engage the relaxed elastic threads to “pinch” the elastic threads therebetween. In other embodiments, anchoring bonds are formed when sections of the web layers around the elastic threads are melted and at least partially encapsulate the elastic threads. Anchoring bonds may be formed as pairs of bonds positioned on opposing sides of a given elastic thread, or as a singular bond that spans at least the width of the elastic thread.

The anchoring bonds are visually present in the pant product and it is desirable for these bonds to be arranged in a visually pleasing manner. In the waistband area, since each elastic thread must be secured to the waistband web layers in multiple locations by the anchoring bonds, the manufacturer is limited on the designs that can be formed with the pattern of bonds.

### BRIEF DESCRIPTION OF THE DRAWINGS

FIG. 1 is a representative isometric view of a finished disposable pant product according to an example embodiment;

FIG. 2 is a schematic representation of a system for manufacturing the pant product of FIG. 1;

FIG. 3 is a simplified representation shown in a plan view of pant products during the steps of manufacture;

FIG. 4 is a plan view of a representative pant product;

FIGS. 4A, 4B, 4C, and 4C′ are cross-sectional views of portions of embodiments the pant product of FIG. 4, with the spacing between the web layers exaggerated for purposes of clarity;

FIG. 5 is a perspective view of an anvil used in the system according to a first embodiment; and

FIGS. 6-10 are partial top plan views of embodiments of the anvil.

Like reference symbols in the various drawings indicate like elements.

### DETAILED DESCRIPTION

The following describes one or more example embodiments of the disclosed disposable pant products and systems and methods for manufacturing same, as shown in the accompanying figures of the drawings described briefly above. Various modifications to the example embodiments may be contemplated by one of skill in the art.

In the embodiments discussed herein, representations of directions or orientations (e.g., up, down, front, forward, side, upper, lower, distal, rear, inner, and outer) may be used for explaining the structure and configurations of the various elements, but these representations should be considered as relative to one another in the particular examples and should not be considered as absolute limitations. Additionally, the discussion below may reference a material or product-based coordinate system with respect to continuous webs in the primary direction of movement, e.g., with a longitudinal orientation being parallel to the direction of movement at a respective location, a lateral orientation being “side-to-side”, perpendicular to the longitudinal orientation within the plane of the product, and a transverse orientation being perpendicular to the longitudinal and lateral orientations. In other instances, the discussion below may reference an absolute coordinate system relative to the particular orientation of the machine (e.g., x, y, and z).

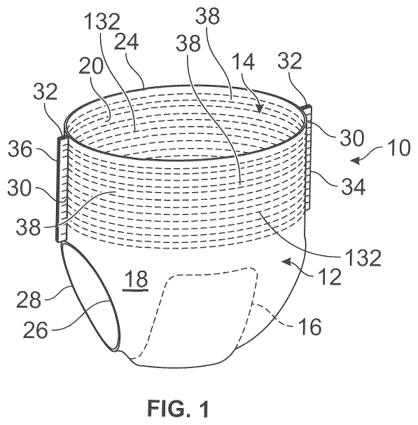

FIG. 1 depicts a disposable pant product 10. Generally, the disposable pant product 10 is formed from a first waistband 12, a second waistband 14, and a core 16 that also collectively define a first (or inner) face (or surface) 18 and a second (or outer) face (or surface) 20. Typically, the inner face 18 is oriented towards the user when wearing the pant product 10 and the outer face 20 is orientated away from the user when wearing the pant product 10. The first and second waistbands 12, 14 have upper edges 22, 24 that define a waist opening and leg contours 26, 28 (which are only shown on one side of the pant product 10 in FIG. 1) that form the leg openings proximate to the core 16. The first and second waistbands 12, 14 have opposite side edges 30, 32 which extend between the upper edges 22, 24 and leg contours 26, 28. The side edges 30 of the first and second waistbands 12, 14 are bonded together as described herein to form a first side seam 34, and the side edges 32 are bonded together as described herein to form a second side seam 36. A waistband area 38 above each of the leg contours 26, 28 are bonded together as described herein.

The first and second waistbands 12, 14 may be formed from any suitable type of material, such as a nonwoven or woven fabric material. The core 16 may be formed from one or more absorbent materials, such as pulp, paper, polymer, or tissue materials and may include additional layers, such as a topsheet (e.g., for skin contact), an acquisition layer (e.g., to facilitate wicking), a backsheet, a debulked structure, and/or embossed pattern. In other examples, the core may have only a single layer, or may have more than two primary layers.

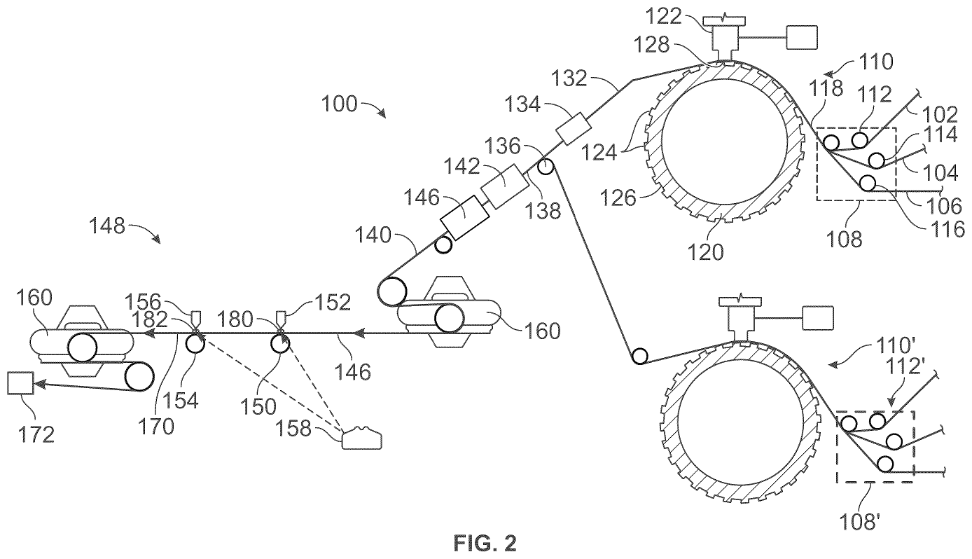

Referring now to FIG. 2, a portion of an exemplary manufacturing line 100 is illustrated. As shown, a first continuous web layer 102, a plurality of continuous elastic threads 104 and a second continuous web layer 106 are fed by a roller apparatus 108, which may include one or more rollers, to a bonding apparatus 110. The elastic threads 104 travel under tension from a creel apparatus (not shown) or similar device. Elastic threads 104 may have any suitable cross-sectional shape that facilitates formation of an elastic composite structure having desired elasticity, visual aesthetic, and manufacturability. As non-limiting examples, elastic threads 104 may have a cross-sectional shape that is round, rectangular, square, or irregular as may be the case where each elastic thread 104 is a multifilament product. The elastic threads 104 may be composed of any suitable elastic material including, for example, sheets, threads or ribbons of thermoplastic elastomers, natural or synthetic rubber, or elastic threads, as non-limiting examples. Each elastic thread 104 may be provided in the form of an individual elastomeric strand or be a manufactured multifilament product that includes many individual elastomeric filaments joined together, such as by a dry-spinning manufacturing process, to form a single, coalesced elastic thread 104.

The roller apparatus 108 includes guide rollers that are employed to accurately position and (optionally) tension the elastic threads 104 and the first and second web layers 102, 106 as they travel toward the bonding apparatus 110. Roller apparatus 108 includes an upper roller 112, a lower roller 116, and a strand guide roller 114 that guide a first assembly 118 that includes the first web layer 102, the second web layer 106, and the elastic threads 104 into the bonding apparatus 110. It is contemplated that rollers 112, 114, 116 may be replaced with other known types of feeding apparatuses and/or replaced by a single roller unit or other known type of feeding apparatus in an alternative embodiment.

After the second web layer 106 is brought next to the first web layer 102 and elastic threads 104, the first assembly 118 is joined together by the bonding apparatus 110. Bonding apparatus 110 may be any known mechanical bonding system including, as non-limiting examples, a thermal welding system, a pressure welding system, a rotary ultrasonic welding system, or a blade ultrasonic welding system. In the illustrated embodiment, bonding apparatus 110 includes a rotary anvil 120 and an ultrasonic fixed blade horn 122, also known as a sonotrode, which cooperate with each other to bond (i.e., fuse) the first web layer 102 to the second web layer 106. Alternative embodiments may include multiple fixed blade horns or one or more rotary horns.

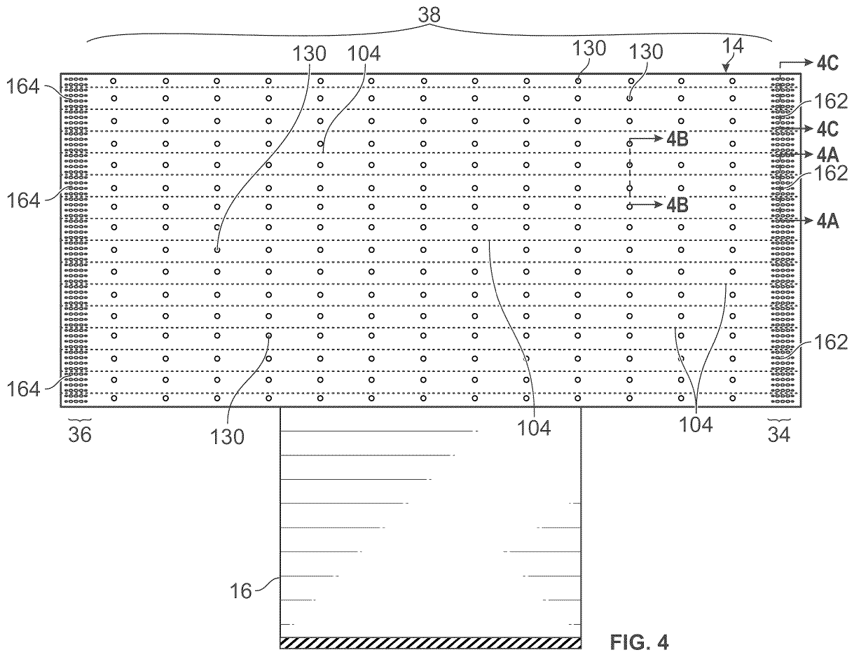

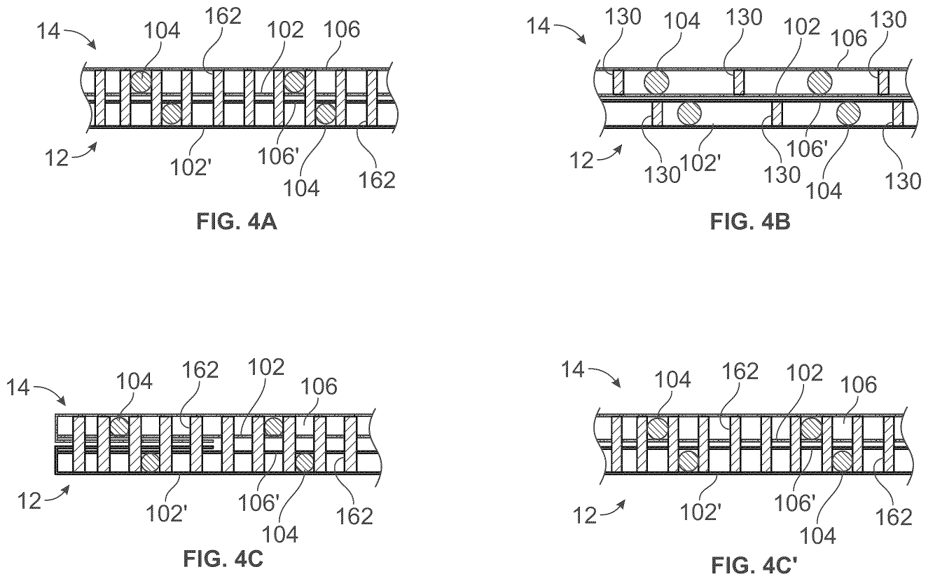

While first web layer 102 and second web layer 106 are depicted and described herein as physically separate components, it is contemplated that alternative embodiments may utilize a unitary web structure that is folded to capture the elastic threads 104 between upper and lower layers of the unitary web structure. In such an embodiment, the portion of the unitary structure positioned below the elastic threads 104 would be referred to as the first web layer 102 and the portion of the unitary structure positioned above the elastic threads 104 would be referred to as the second web layer 106. For example, a folding apparatus (not shown) may be included to fold a portion of the unitary web structure over another portion of the unitary web structure to overlap the elastic threads 104. A product with a folded waist edge may be alternatively be formed with a folding apparatus (not shown) that folds a waist edge portion of one of the first web layer 102 over and around the waist edge second web layer 106 to create a folded waist edge as shown in FIG. 4C. FIG. 4C′ depicts an alternative embodiment that does not include a folded waist edge.

The pant product 10 may include a multi-piece chassis, wherein the front and/or rear waistbands include two or more web pieces joined together. In such embodiments, additional web layers (not shown) may be joined with first and second web layers 102, 106 at a location upstream of the bonding apparatus 110 and the subsequently fed together with first and second web layers 102, 106 into the bonding apparatus 110, with the elastic threads 104 positions between first and second web layers 102, 106 and the additional one or more web layers.

The anvil 120 and the horn 122 are illustrated according to one embodiment of the disclosure. As shown, the anvil 120 includes an arrangement of discrete projections 124 that extend outward from the anvil face 126. Projections 124 may have planar working surfaces, planar side surfaces, or some mixture of curved and straight working and side surfaces in alternative embodiments. The working surfaces of each projection 124 may have a smooth or substantially smooth surface contour or an arrangement of grooves. The working surface 128 of the horn 122 has either a smooth or substantially smooth surface contour or an arrangement of grooves formed within the working surface 128. In either case, working surface 128 cooperates with the projections 124 on the anvil face 126 to form discrete bonds or welds 130.

These projections 124 are constructed to form bonds 130 which fuse the first and second web layers 102, 106 together, as graphically depicted in FIG. 4B. The projections 124 may include any number of projections 124 and may be arranged in any number of alternative configurations to achieve a desired pattern of waistband bonds 130 on the finished pant product 10 which may be anchoring bonds or laminating bonds. In an embodiment, anchoring bonds are formed when the elastic threads 104 are frictionally engaged by the adjacent bonds when the elastic threads 104 are relaxed. In an embodiment, anchoring bonds are formed when the projections 124 engage the web layers 102, 106 on opposites sides of the elastic threads 104 which causes the sections of the web layers 102, 106 proximate the elastic threads 104 to melt and at least partially encapsulate the elastic threads 104 and/or fictionally engage the elastic threads 104 in position relative to the web layers 102, 106. Laminating bonds are formed when only the web layers 102, 106 are bonded together and the elastic threads 104 are not engaged by the bonds. In an embodiment, the projections 124 may be arranged so that the elastic threads 104 are between a first row of projections 124 on one side of the elastic threads 104 which form laminating bonds and a second row of projections 124 on the opposite side of the elastic threads 104 which form laminating bonds such that elastic threads 104 are between the rows of the projections 124. Projections 124 forming anchoring bonds may be provided in addition to projections 124 forming laminating bonds. In another embodiment, the projections 124 may be arranged so that each elastic thread 104 passes between two adjacent projections 124 on the face 126 of the anvil 120 and the resulting bonds form anchoring bonds or form laminating bonds. The respective working surfaces of projections 124 may be configured to form bonds of similar size and shape, or bonds of different size and/or shape in alternative embodiments. As non-limiting examples, respective land surfaces of projections 124 may be circular, rectangular, crescent shaped, or have irregular shapes that may be selected to form a desired overall pattern on the finished pant product 10. In an embodiment, bonding apparatus 110 may be replaced with an adhesive system (not shown) that applies adhesive to the elastic strands and/or one or both web layers 102, 106.

During the manufacturing process, the first and second web layers 102, 106 are positioned between the face 126 of the anvil 120 and the working surface 128 of the horn 122. Elastic threads 104 are positioned between the first and second web layers 102, 106 in a tensioned state. The position of horn 122 is controlled to maintain a nip gap between the working surface 128 of horn 122 and land surfaces of the projections 124. Bonding apparatus 110 may include any known positioning means that exerts a force on at least one of the horn 122 and the anvil 120 to maintain the desired nip gap. The bonding apparatus 110 forms bonds 130 thereon, see FIG. 4, and forms a second assembly 132.

In an embodiment, the second assembly 132 travels downstream to a slitting apparatus 134 which separates the second assembly 132 into the first waistband 12 and the second waistband 14 which sit side-to-side. Alternatively, the slitting apparatus 134 is eliminated and the roller apparatus 108 and bonding apparatus 110 form the first waistband 12 and a second roller apparatus 108′ and a second bonding apparatus 110′ form the second waistband 14 which are combined together at a roller 136 such that the first and second waistbands 12, 14 are side-to-side. This forms a third assembly 138.

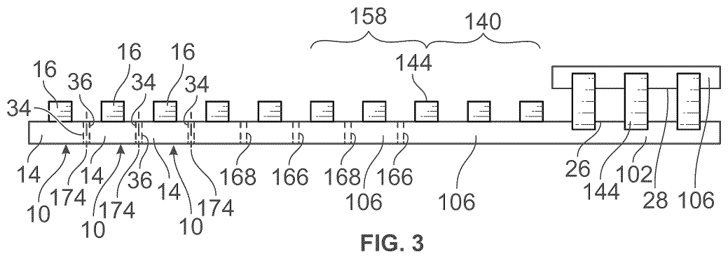

Thereafter, the third assembly 138 passes through known apparatuses to form a fourth assembly 140. The apparatuses include, but are not limited to, an insert placement apparatus 142 which places an absorbent insert 144 onto the first web layers 102 that forms the core 16, and a folding apparatus 146 which folds the absorbent insert 144 and overlaps the first and second waistbands 12, 14 such that the first web layers 102 face each other, see FIG. 3. An optional die cutting apparatus (not shown) which forms leg contours may also be provided in apparatuses.

Thereafter, the fourth assembly 140 passes through an ultrasonic side seam-bonding apparatus 148. The ultrasonic side seam bonding apparatus 148 includes a first anvil 150 and its ultrasonic horn(s) 152, and, optionally, a one or more additional anvil(s) and corresponding ultrasonic horn(s). Each anvil 150 is provided with one or more anvil inserts 158 having a predetermined profile. In embodiments that include multiple anvil/horn stations, the anvils 150 are laterally spaced apart inline and in a machine direction. The ultrasonic horn(s) 152 apply vibration energy to the fourth assembly 140 simultaneously and in cooperation with a respective anvil 150 to form anchoring bonds or welds 162, 164 in respective side edge portions 166, 168 of the fourth assembly 140 that will subsequently form the side seams 34, 36 of an individual finished pant product 10 and thereby form a fifth assembly 170. In an embodiment, the bonds 130 are also within the side edge portions 166, 168 such that the anchoring bonds 162, 164 are in addition to the bonds 130 within the side edge portions 166, 168. In another embodiment, the anvil 120 does not form the bonds 130 in the areas which eventually form the side edge portions 166, 168 such that only anchoring bonds 162, 164 are within the side edge portions 166, 168. In an embodiment, bonds 130 are spaced further apart from one another than bonds 162, 164. In other embodiments bonds 130 are spaced closer together than bonds 162, 164 or the inter-bond spacing of bonds 130 and bonds 162, 164 is equidistant. In some embodiments, an optional adhesive system (not shown) may be positioned upstream of the ultrasonic side seam bonding apparatus 148 to apply adhesive to at least a portion of the side edge portions 166, 168.

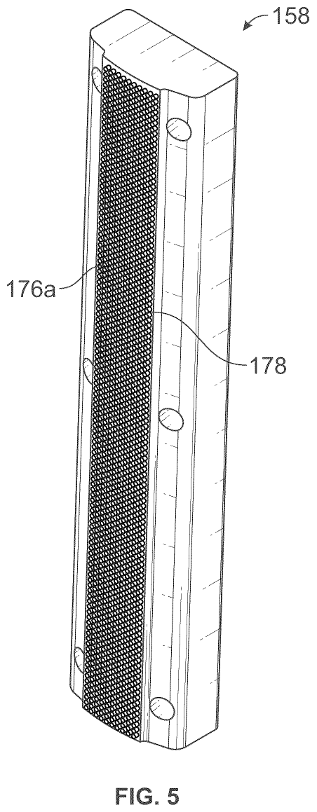

The fifth assembly 170 then passes through a cutting apparatus 172 which forms cut lines 174 between the side edge portions 166, 168 to separate the fifth assembly 170 into the individual pant products 10. The cutting apparatus 172 may be a cutting or knife unit which the separates the continuous web into discrete pant products 10 at a location between the side edge portions 166, 168. While side edge portions 166, 168 are depicted as two distinct bond regions positioned on opposing sides of cut lines 174 in FIG. 3, side edge portions 166, 168 may be one continuous bond region that spans a respective cut line 174 in embodiments where projections 176 form a continuous pattern across the anvil insert 158, as further described below, and illustrated in FIGS. 5-7 and FIG. 10.

As such, the manufacturing line 100 operates to form finished pant products 10 from the various continuous (and/or discrete) layers of materials.

The insert 158 of each anvil 150 includes an arrangement of discrete projections 176 that extend outward from the anvil face 178. The upper face of the projections 176 may have flat or substantially flat working surfaces 177, as shown in the embodiments of FIGS. 5-9, or may include a plurality of grooves 188 extending downward into the body of the projection 176, as shown in FIG. 10. Working surface(s) 180 of the horn(s) 152 are flat or have a substantially smooth surface contour in one non-limiting embodiment. Alternatively, working surface(s) 180 may include an arrangement of projections that interact the pattern of projections 176 on the anvils 150.

The projections 176 are constructed to form anchoring bonds 162 which fuse the first web layer 102, the second web layer 106, and the elastic threads 104 together in each side edge portion 166, 168 as shown in FIG. 4A. The projections 176 may include any number of projections 176 arranged in any number of alternative configurations to achieve a desired pattern of side seam bonds on the finished pant product 10. The projections 176 may have a planar working surface, planar side surfaces, or some mixture of curved and straight working and side surfaces in alternative embodiments. The projections 176 form at least one anchoring bond pair, and preferably at least three anchoring bond pairs, along the length of each of the elastic threads 104 in each side edge portion 166, 168. Bonding each elastic thread at least three times in each side edge portion 166, 168 provides additional structure to prevent the snap back of the respective elastic thread 104 into the area (which forms waistband area 38) between the side edge portions 166, 168.

During the manufacturing process, the first waistband 12 and the second waistband 14 are positioned between the face 178 of each anvil 150 and the working surface 180 of the horn(s) 152. The elastic threads 104 are still in the tensioned state. The position of horn(s) 152 is controlled to maintain a nip gap between the working surface 180 of horn(s) 152 and land surfaces of the projections 176. Side seam bonding apparatus 148 may include any known positioning means that exerts a force on at least one of the horn(s) 152 and the anvil 150 to maintain the desired nip gap.

The respective working surfaces 177 of projections 176 may be configured to form bonds of similar size and shape, or bonds of different size and/or shape in alternative embodiments. As non-limiting examples, respective working surfaces 177 of projections 176 may be circular, rectangular, crescent shaped, or have irregular shapes that may be selected to form a desired overall pattern on the finished pant product 10.

The anchoring bonds 162, 164 in the side edge portions 166, 168 are sized and positioned to ensure a proper anchoring of each elastic thread 104 and may be more closely spaced than the spacing of the bonds 130 formed in the area between the side edge portions 166, 168 (which forms waistband area 38). Since the anchoring bonds 162, 164 in the side edge portions 166, 168 (which form side seams 34, 36) secure the elastic threads 104 to the first and second web layers 102, 106, the bonds 130 in the area (which forms waistband area 38) between the side edge portions 166, 168 can have any desired patterning. Therefore, the manufacturer can choose to either use anchoring bonds and/or laminating bonds in the area between the side edge portions 166, 168 (which forms waistband area 38) and therefore can create a variety of visually pleasing patterns of bonds.

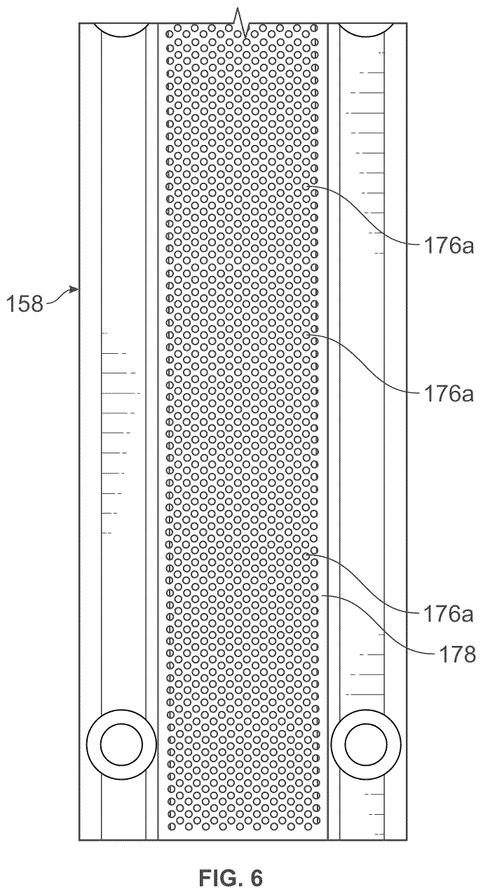

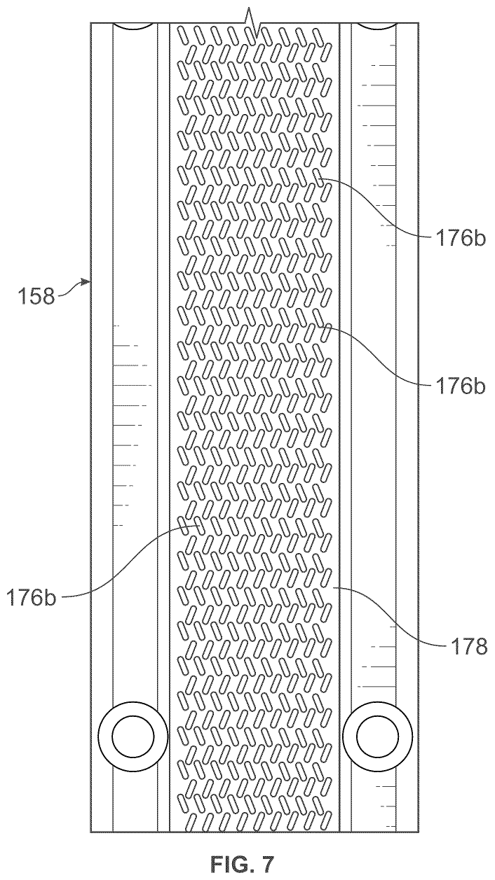

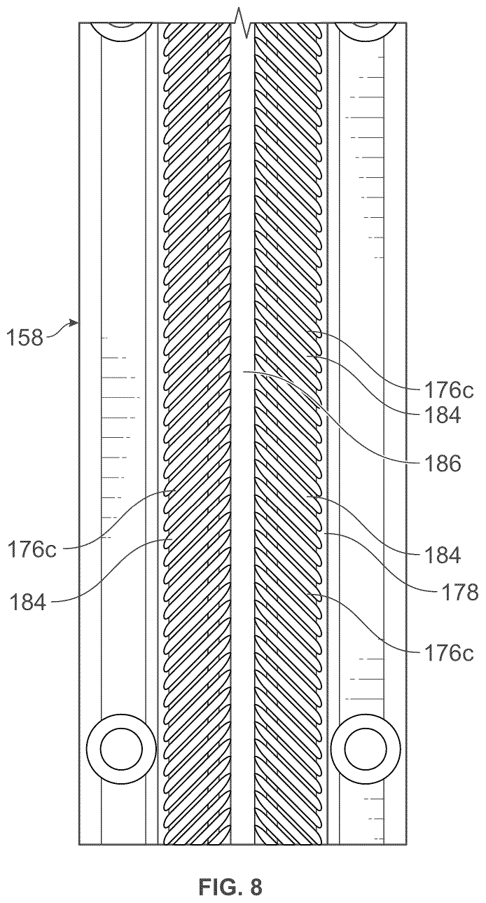

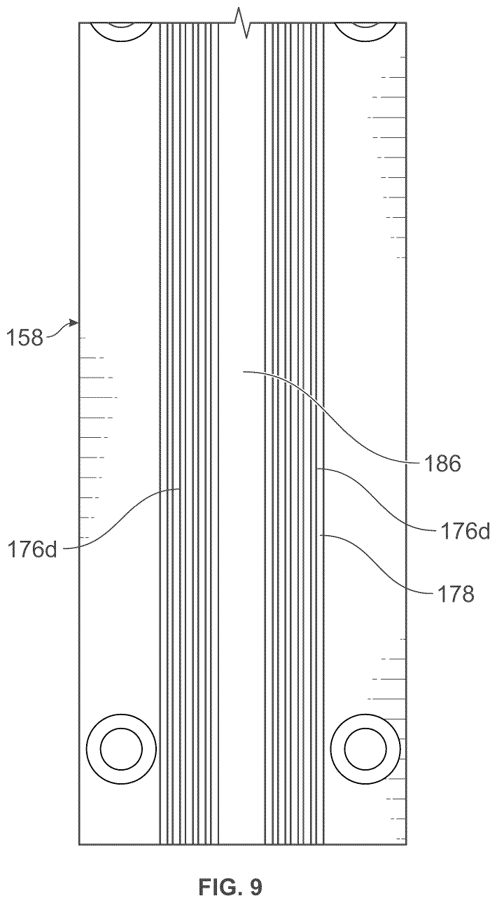

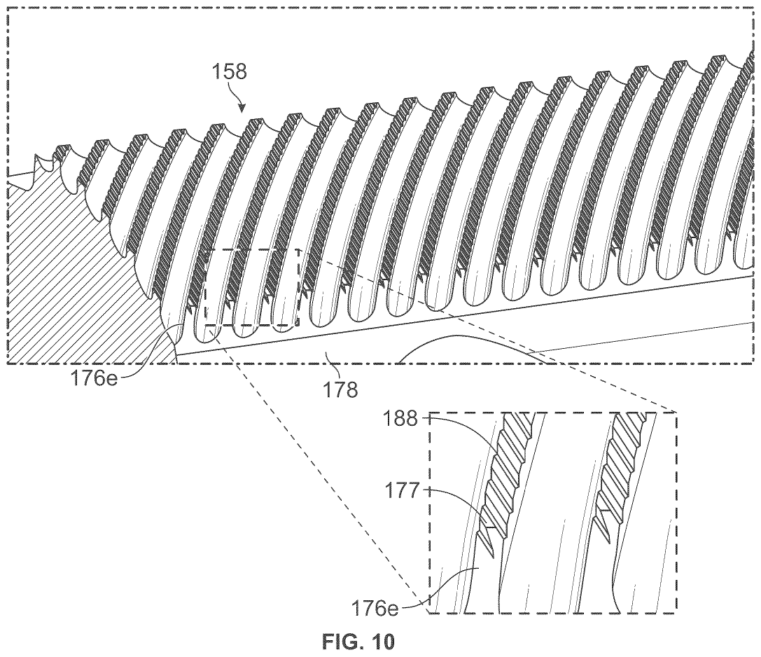

FIGS. 5-9 show embodiments of patterns of the anchoring projections 176a, 176b, 176c, 176d provided on the insert(s) 158 for the anvil(s) 150. In FIGS. 5 and 6, the anchoring projections 176a are array of raised circles which are in offsetting rows and columns. In FIG. 7, the anchoring projections 176b are an array of raised elongated bars which are in offsetting rows and columns, with the bars being angled relative to each other in adjacent rows. In FIG. 8, the projections 176c form columns of elongated grooves 184 which are angled relative to a longitudinal direction that the elastic threads 104 pass such that each elastic thread 104 passes over three of the projections 176c. In FIG. 9, the projections 176d are continuous bars that extend across the length of the insert 158. When paired with a grooved horn, the anchoring projections 176a-176d of FIGS. 5-9 form bonds by melting sections of the web layers 102, 106 proximate the elastic threads 104 and at least partially encapsulating the elastic threads 104 and/or frictionally lock the elastic threads 104 in position relative to the web layers 102, 106.

In the embodiments of FIGS. 5-9, the working surfaces 177 of the projections 176a-176d are smooth or substantially smooth, meaning that they do not include any internal grooves or notches. These projections 176a-176d may be paired with a horn with a grooved working surface such that the interaction between the horn and each projection 176a-176b forms a pattern of multiple anchoring bonds 162, 164, some of which form pairs located on opposing sides of a respective elastic strand 104, to anchor the strand 104 in position within the side seam 34, 36. Alternately, projections 176a-176d may be paired with a horn with a smooth working surface such that the interaction between the smooth horn and each projection 176a-176b forms a single anchoring bond 162, 164, that spans across the width of one or more elastic strands 104 to anchor the strand 104 in position within the side seam 34, 36.

In the embodiment of FIG. 10, on the other hand, the working surfaces 177 of projections 176e include grooves 188 that extend downward into the height of the projections 176e. This style of grooved projections could be paired either with a horn with a smooth working surface or a horn with a grooved working surface to form a pattern of anchoring bonds 162, 164, some of which form pairs located on opposing sides of a respective elastic strand 104, to anchor the strand 104 in position within the side seam 34, 36. Projections 176e are shown in FIG. 11 arranged as an array of raised elongated bars that are angled relative to the central longitudinal axis of the insert 158.

In general, embodiments may include projections with or without internal grooves. The projections may have the overall shape of shorter angled bars, similar to those of FIG. 7, arcs, curved or chevron-shaped bars generally extending along the length of the insert, dots, angled bars, horizontal bars, vertical bars, or any other circular, rectangular, crescent shaped, or irregular shapes. Embodiments of projections may be arranged in a pattern of at least one of dots, vertical lines, horizontal lines, angled lines, and curved lines. Horn 152 interacts with the plurality of projections to form a plurality of discrete bonds corresponding to each dot or line of the pattern.

The embodiments shown in FIGS. 8 and 9 include a central section 186 which is devoid of projections 176c. This provides an area where the side edge portions 166, 168 can be easily separated from one another by the cutting apparatus 172. Such a central section 186 can be provided in the inserts 158 shown in FIGS. 5-7 and 10. With such a central section 186, the bonds 162, 164 are not along the outer edges of the resulting side seams 34, 36 which provides a softer feel along the edges thereof. In alternative embodiments, the projections 176c, 176d of FIGS. 8 and 9 may be reconfigured to span across central section 186 thus defining a continuous pattern of projections and continuous pattern of bonds 162, 164 that extend across the entire width of the side seams 34, 36.

In the embodiments shown in FIGS. 5-10, the interaction between the horn(s) 152 and the anchoring projections 176a, 176b form bonds 162, 164 which serve the dual function of creating side seams 34, 36 that fuse together first and second waistbands 12, 14 and anchor the elastic threads 104 in place within the side seam 34, 36 when the pants 10 are cut into discrete pieces and the elastic threads 104 are relaxed.

The detailed description herein describes exemplary embodiments and is not intended to be limited to the expressly disclosed combination(s). Therefore, unless otherwise noted, features disclosed herein may be combined together to form additional combinations that were not otherwise shown for purposes of brevity. As such, references to a feature or aspect are intended to describe a feature or aspect of an example, not to imply that every embodiment thereof must have the described feature or aspect. Furthermore, it should be noted that the description illustrates a number of features. While certain features have been combined together to illustrate potential system designs, those features may also be used in other combinations that are not expressly disclosed. Thus, the depicted combinations are not intended to be limiting, unless otherwise noted.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, unless otherwise limited or modified, lists with elements that are separated by conjunctive terms (e.g., “and”) and that are also preceded by the phrase “one or more of” or “at least one of” indicate configurations or arrangements that potentially include individual elements of the list, or any combination thereof. For example, “at least one of A, B, and C” or “one or more of A, B, and C” indicates the possibilities of only A, only B, only C, or any combination of two or more of A, B, and C (e.g., A and B; B and C; A and C; or A, B, and C).

The description of the present disclosure has been presented for purposes of illustration and description, but it is not intended to be exhaustive or limited to the disclosure in the form disclosed. Many modifications and variations will be apparent to those of ordinary skill in the art without departing from the scope and spirit of the disclosure. Explicitly referenced embodiments herein were chosen and described in order to best explain the principles of the disclosure and their practical application, and to enable others of ordinary skill in the art to understand the disclosure and recognize many alternatives, modifications, and variations on the described example(s). Accordingly, various embodiments and implementations other than those explicitly described are within the scope of the claims.

What is claimed is:

1. A system for manufacturing a disposable pant product comprising:
a first apparatus configured to form side-to-side first and second assemblies, each assembly comprising a plurality of tensioned elastic threads between first and second web layers, wherein the first apparatus couples together the first and second web layers of each of the first and second assemblies;
an insert placement apparatus configured to place an absorbent insert onto the first web layer of each of the first and second assemblies;
a folding apparatus configured to fold the absorbent insert and to overlap the first and second assemblies such that the first web layers face each other and opposite side edge portions of the first and second assemblies align with each other; and
a second apparatus configured to form a plurality of bonds that bond the aligned side edge portions to form side seams and secure the elastic threads in position within the side seams.

2. The system of claim 1, wherein the second apparatus comprises:
an anvil having a plurality of projections defining a side seam pattern; and
an ultrasonic horn having a working surface that interacts with the plurality of projections to form the plurality of bonds; and
wherein at least one of the working surface of the ultrasonic horn and faces of the plurality of projections have grooves formed therein.

3. The system of claim 1, wherein the second apparatus includes a first anvil and ultrasonic horn and a second anvil and ultrasonic horn which is spaced from the first anvil and ultrasonic horn.

4. The system of claim 1, wherein each elastic thread is bonded at least once in each side edge portion.

5. The system of claim 1, wherein each elastic thread is bonded at least three times in each side edge portion.

6. The system of claim 1, wherein the first apparatus forms bonds which are spaced further apart from each other than the bonds which are formed by the second apparatus.

7. The system of claim 1, wherein the second apparatus includes an anvil and an ultrasonic horn; and
wherein the anvil comprises at least one insert having a plurality of projections thereon that form the plurality of discrete bonds.

8. The system of claim 1, wherein the first web layer of each of the first and second assembly are formed from a common continuous first web layer and the second web layer of each of the first and second assembly are formed from a common continuous second web layer, and further comprising a slitting apparatus for slitting the common continuous webs into the first and second assemblies.

9. The system of claim 1, wherein the first apparatus includes a first bonding apparatus configured to form bonds to bond together the first and second web layers of the first assembly and a second bonding apparatus configured to form bonds to bond together the first and second web layers of the second assembly.

10. (canceled)

11. The system of claim 1, wherein the first apparatus comprises an anvil having a plurality of projections thereon and an ultrasonic horn, and
. wherein the first apparatus further bonds the elastic threads to the first and second web layers in each of the first and second assemblies.

12. The system of claim 1, wherein the first apparatus comprises an adhesive applicator that bonds together the first and second web layers.

13. (canceled)

14. A disposable pant product comprising:
a first waistband bonded to a second waistband at first and second side seams and defining a waist opening at an upper edge thereof and a pair of leg openings, wherein the first waistband includes elastic threads between first and second web layers and a pattern of waistband bonds bonding the first and second web layers together, and the second waistband includes elastic threads between first and second web layers and a pattern of waistband bonds bonding the first and second web layers of the second waistband together, wherein the first side seam includes aligned first side edge portions of the first and second waistbands and a pattern of side seam bonds bonding the first side edge portions together and anchoring the elastic threads within the first side seam, and the second side seam includes aligned second side edge portions of the first and second waistbands and a pattern of side seam bonds bonding the second side edge portions together and anchoring the elastic threads within the second side seam; and
an absorbent core positioned between the first web layers of the first and second waistbands.

15. The disposable pant product of claim 14, wherein the pattern of waistband bonds further bonds at least one of the elastic threads of the first waistband to the first and second web layers of the first waistband and at least one of the elastic threads of the second waistband to the first and second web layers of the second waistband.

16. The disposable pant product of claim 14, wherein the pattern of side seam bonds comprises at least one weld anchoring each elastic thread.

17. The disposable pant product of claim 14, wherein the pattern of side seam bonds comprises at least three welds anchoring respective ends of each elastic thread.

18. The disposable pant product of claim 14, wherein the bonds within the pattern of waistband bonds are spaced further apart from each other than the bonds within the pattern of side seam bonds.

19. The disposable pant product of claim 14, wherein the elastic threads are positioned between pairs of discrete bonds of the pattern of side seam bonds that anchor the elastic threads within the first and second side edge portions.

20. The disposable pant product of claim 14, wherein the pattern of side seam bonds comprises a repeating pattern of at least one of dots, vertical lines, horizontal lines, angled lines, and curved lines; and
wherein each dot or line comprises a plurality of bonds.

21. A method of manufacturing a disposable pant product comprising:
forming a first assembly including first and second web layers which are bonded together and having a plurality of elastic threads under tension therebetween;
forming a second assembly including first and second web layers which are bonded together and having a plurality of elastic threads under tension therebetween;
placing an absorbent insert onto the first web layer of each of the first and second assemblies;
folding the absorbent insert and overlapping the first and second assemblies such that the first web layers face each other and opposite side edge portions of the first and second assemblies align with each other; and
bonding the aligned side edge portions to bond the first assembly, the second assembly, and the plurality of elastic threads together such that the plurality of elastic threads are anchored within side seam regions of the disposable pant product by a plurality of discrete bonds.

22. The method of claim 21 wherein at least one of plurality of elastic threads is further bonded to the first and second web layers in the forming steps.

23. The method of claim 21, comprising forming a continuous assembly including a common continuous first web layer and a common continuous second web layer having a plurality of elastic threads under tension therebetween; and
slitting the continuous assembly to form the first assembly and the second assembly.

24. The method of claim 21, comprising mechanically, thermally, or ultrasonically bonding the first assembly, the second assembly, and the plurality of elastic threads together within the side seam regions.

25. The method of claim 21, comprising bonding the first assembly, the second assembly, and the plurality of elastic threads together within the side seam regions with a pattern of side seam bonds comprising a repeating pattern of at least one of dots, vertical lines, horizontal lines, angled lines, and curved lines; and
wherein each dot or line comprises a plurality of bonds.

26. The method of claim 21, comprising bonding the first and second web layers of the first assembly and the first and second web layers of the second assembly with a pattern of waistband bonds; and
bonding the aligned side edge portions with a side seam pattern of bonds;
wherein bonds within the pattern of waistband bonds are spaced further apart from each other than bonds within the pattern of side seam bonds.

27. The method of claim 21, comprising bonding the first and second web layers of the first assembly and the first and second web layers of the second assembly with adhesive.
